(19) United States
(12) Patent Application Publication
(10) Pub. No.: US 2023/0355079 A1
(43) Pub. Date: Nov. 9, 2023

(54) ENDOSCOPE

(71) Applicant: FUJIFILM Corporation, Tokyo (JP)

(72) Inventor: Takashi HARADA, Kanagawa (JP)

(73) Assignee: FUJIFILM Corporation, Tokyo (JP)

(21) Appl. No.: 18/353,845

(22) Filed: Jul. 17, 2023

Related U.S. Application Data
(63) Continuation of application No. PCT/JP2021/045672, filed on Dec. 10, 2021.

(30) Foreign Application Priority Data
Jan. 26, 2021 (JP) 2021-010058

(51) Int. Cl. A61B 1/00

(52) U.S. Cl. CPC A61B 1/00098; A61B 1/00103

(57) ABSTRACT

The endoscope includes an insertion part to be inserted into a subject, a distal end part main body, a treatment tool contact portion, and an elevator. The distal end part main body has an elevator housing space communicating with a treatment tool outlet port. The treatment tool contact portion is provided in the elevator housing space and comes into contact with the treatment tool to control an advancing direction of the treatment tool. The elevator is provided in the elevator housing space and elevates the treatment tool of which the advancing direction is controlled by the treatment tool contact portion.

### CROSS-REFERENCE TO RELATED APPLICATIONS

This application is a Continuation of PCT International Application No. PCT/JP2021/045672 filed on 10 Dec. 2021, which claims priority under 35 U.S.C § 119(a) to Japanese Patent Application No. 2021-010058 filed on 26 Jan. 2021. The above application is hereby expressly incorporated by reference, in its entirety, into the present application.

### BACKGROUND OF THE INVENTION

### 1. Field of the Invention

The present invention relates to an endoscope comprising an elevator for a treatment tool at a distal end part of an insertion part.

### 2. Description of the Related Art

In recent years, in the medical field, diagnosis, treatment, or the like using an endoscope has become increasingly popular. The endoscope comprises an insertion part to be inserted into a subject and an operation part consecutively provided at a proximal end part of the insertion part. The operation part is provided with a treatment tool inlet port (forceps port) for introducing various treatment tools. The introduced treatment tool is used for treatment or the like of an observation target by being led out of a treatment tool outlet port that is open at a distal end part of the insertion part and protruding outside the distal end part. In a side-viewing endoscope, in the distal end part, the treatment tool outlet port through which the treatment tool protrudes in a direction perpendicular to an axial direction of the insertion part is provided, and an elevator that comes into contact with the treatment tool to elevate the treatment tool and that is used to lock the treatment tool is provided in the distal end part.

The operation part is provided with an elevating operation lever, a bending operation knob, or the like as an operation input part. By operating the elevating operation lever, a position state such as reclining or elevating the elevator provided at the distal end part is operated. With this, the treatment tool is elevated in a direction of the treatment tool outlet port by being supported by the elevator, or the elevated treatment tool is locked by, for example, being sandwiched and pressed down by the elevator and the distal end part main body forming the treatment tool outlet port.

Various shapes of the elevator are disclosed. For example, a lifter that can lock a treatment tool or the like without adverse influence, such as damage to the treatment tool or squeezing of a catheter is disclosed (JP2008-536579A, corresponding to US2006/235271A1). The lifter has a grip distal end consisting of a polymer material.

### SUMMARY OF THE INVENTION

In a case where the treatment tool is elevated by the elevator, the treatment tool is led out of the treatment tool outlet port, and the elevator is operated from a reclined position to an elevated position. By this operation, the treatment tool is supported by the elevator and is elevated in response to the elevation of the elevator. In a case where the treatment tool is elevated by the operation, the treatment tool is bent near the treatment tool outlet port, and a contact point between the treatment tool and the upper part or the like of the treatment tool outlet port provided in the distal end part main body serves as a fulcrum point, and a contact point between the treatment tool and the elevator serves as a force point, so that the treatment tool enters an elevated state.

For example, as shown in FIG. 16, in a case where a treatment tool insertion channel 61 is formed in a state close to horizontal to an axial direction Ax of the insertion part in a treatment tool outlet port 62 provided in a distal end part main body 67, a distance D1 between a fulcrum point 65 and a force point 66 described above can be made relatively long, so that a small amount of force is sufficient to operate an elevator 63 to elevate a treatment tool 64. In addition, this facilitates control of a lead-out direction of the treatment tool 64. Therefore, in this case, it can be said that an elevating force of the elevator 63 is high.

Meanwhile, in this case, since there is a probability that the distal end of the treatment tool 64, which has come out of the treatment tool outlet port 62, and the elevator 63 may come into contact with each other at an angle close to perpendicular, the advancement of the treatment tool 64 may be hindered, which may cause a risk of making it difficult for the treatment tool 64 to protrude outside the distal end part. In order to avoid this, it is necessary to lower (recline to the reclined position) the elevator 63 more than necessary. Therefore, there is a probability of a problem occurring with the insertability of the treatment tool.

On the other hand, in order to improve the insertability of the treatment tool, for example, as shown in FIG. 17, in a case where the treatment tool insertion channel 61 is formed with a tilt near the treatment tool outlet port 62 in a direction in which the treatment tool 64 protrudes outside, the distal end of the treatment tool 64, which has come out of the treatment tool outlet port 62 provided in the distal end part main body 67, and the elevator 63 come into contact with each other with a tilt, which improves the insertability of the treatment tool 64. However, since a distance D2 between the fulcrum point 65 and the force point 66 described above is shorter than the distance D1 in a case where the treatment tool 64, which has come out of the treatment tool outlet port 62, is elevated by the elevator 63, there is a probability that a greater force may be required to operate the elevator 63 to elevate the treatment tool 64, which may cause a risk of making it difficult to operate the elevator 63 to control the lead-out direction of the treatment tool 64. Therefore, in this case, there is a probability that the elevating force may decrease.

An object of the present invention is to provide an endoscope capable of improving both an insertability and an elevating force of a treatment tool at an endoscope distal end part.

The present invention relates to an endoscope comprising: an insertion part to be inserted into a subject; a distal end part main body that is located at a distal end of the insertion part, has a distal end surface provided with a treatment tool outlet port, and forms an elevator housing space communicating with the treatment tool outlet port; a treatment tool contact portion that is provided in the elevator housing space and comes into contact with a treatment tool to control an advancing direction of the treatment tool; and an elevator that is provided in the elevator housing space and elevates the treatment tool of which the advancing direction is controlled by the treatment tool contact portion.

It is preferable that one end of the elevator is supported at an elevator support portion so as to be rotationally movable with respect to the distal end part main body, and that the treatment tool contact portion is provided in a vicinity of the elevator support portion.

It is preferable that the treatment tool contact portion is provided in a half region of the elevator on an elevator support portion side in a longitudinal direction.

It is preferable that a cap that is attached to the distal end part main body and covers at least a part of at least the elevator housing space is further provided, and that the treatment tool contact portion is provided on the cap.

It is preferable that the treatment tool contact portion is provided on the distal end part main body.

It is preferable that the treatment tool contact portion is a protrusion.

It is preferable that the treatment tool contact portion has a triangular prism shape.

It is preferable that a plurality of the treatment tool contact portions are provided.

It is preferable that the elevator includes a distal end part protrusion in a half region opposite to an elevator support portion side in a longitudinal direction.

It is preferable that the treatment tool contact portion controls the advancing direction of the treatment tool to a direction of the distal end part protrusion in a case where the elevator is in a reclined position, and that the elevator includes the distal end part protrusion at a position where the treatment tool comes into contact in a case where the elevator is elevated to an elevated position.

It is preferable that a part or all of the endoscope is disposable.

According to the present invention, in the endoscope comprising the elevator, both the insertability and the elevating force of the treatment tool can be improved.

### BRIEF DESCRIPTION OF THE DRAWINGS

FIG. 1 is a perspective view showing an outline of an endoscope system.

FIG. 2 is a perspective view of a distal end cap and a distal end part main body.

FIG. 3 is a perspective view of the distal end part consisting of the distal end cap and the distal end part main body.

FIG. 4 is a perspective view of the distal end part main body.

FIG. 5 is a plan view of the distal end part main body.

FIG. 6 is a perspective view of an elevator provided with a treatment tool contact portion.

FIG. 7 is a perspective view of the distal end cap provided with the treatment tool contact portion.

FIGS. 8A and 8B are each a perspective view of the distal end part main body provided with the treatment tool contact portion, in which FIG. 8A is a perspective view seen from one side, and FIG. 8B is a perspective view seen from the other side different from FIG. 8A.

FIG. 9 is a perspective view of an elevator provided with a treatment tool contact portion whose inclination is adjusted.

FIG. 10 is a perspective view of an elevator provided with a treatment tool contact portion having a triangular prism shape.

FIG. 11 is a perspective view of an elevator provided with a plurality of the treatment tool contact portions.

FIG. 12 is a perspective view of an elevator provided with the treatment tool contact portion and a distal end part protrusion.

FIG. 13 is a cross-sectional view of a main part of a distal end part showing an example of a relationship between the elevator, which is provided with the treatment tool contact portion and the distal end part protrusion, and a treatment tool.

FIG. 14 is a cross-sectional view of a main part of a distal end part showing another example of a relationship between the elevator, which is provided with the treatment tool contact portion and the distal end part protrusion, and the treatment tool.

FIG. 15 is a flowchart of a step of performing treatment with the treatment tool using an endoscope.

FIG. 16 is a cross-sectional view of a main part of a distal end part showing an example of a relationship between a conventional treatment tool channel, treatment tool, and elevator.

FIG. 17 is a cross-sectional view of a main part of a distal end part showing another example of the relationship between the conventional treatment tool channel, treatment tool, and elevator.

### DESCRIPTION OF THE PREFERRED EMBODIMENTS

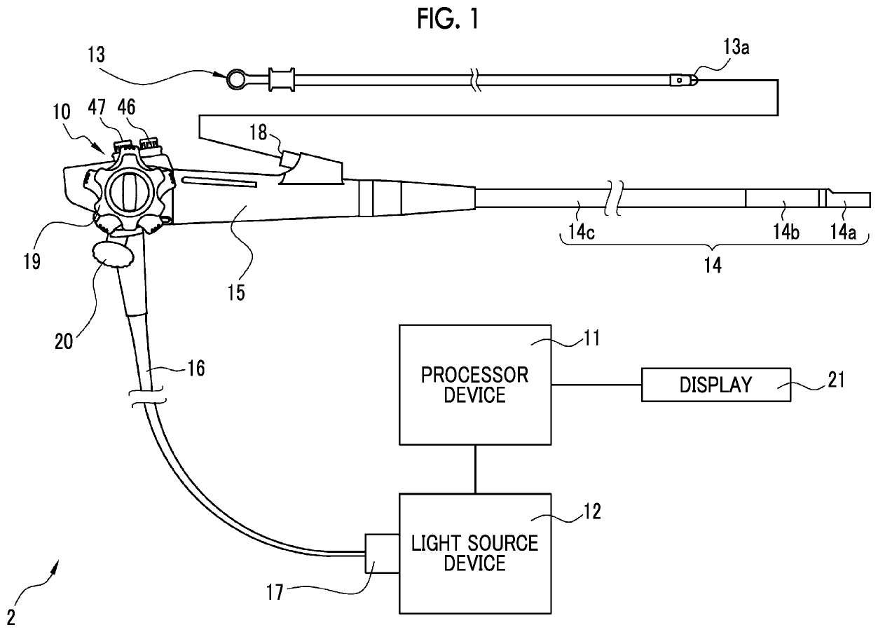

As shown in FIG. 1, an endoscope system 2 comprises an endoscope 10, a processor device 11, a light source device 12, a treatment tool 13, and a display 21. The endoscope 10 has an insertion part 14 to be inserted into a body as a subject, an operation part 15 consecutively provided at the proximal end part of the insertion part 14, and a universal cord 16 connected to the processor device 11 and the light source device 12. A connector 17 is attached to the distal end of the universal cord 16. The endoscope 10 is communicably connected to the processor device 11 via the light source device 12 that is connected to the connector 17.

The insertion part 14 is divided into a distal end part 14a, a bending part 14b, and a flexible tube part 14c in order from the distal end. The bending part 14b is consecutively provided on the proximal end side of the distal end part 14a and is configured by coupling a plurality of bending pieces. The flexible tube part 14c has flexibility and is provided from the proximal end side of the bending part 14b to the coupling part of the operation part 15.

The endoscope 10 is a side-viewing endoscope that is mainly used as a duodenal endoscope. In this type of endoscope, the insertion part 14 is inserted into the subject via the oral cavity and is inserted from the esophagus to the duodenum through the stomach. In the side-viewing endoscope, the treatment tool 13 is inserted into the common bile duct or the like from the biliary tract of the duodenum and performs a treatment, such as a predetermined examination or treatment. Specific examples of the treatment tool 13 include a biopsy forceps having a cup capable of collecting biological tissue, a knife for endoscopic sphincterotomy (EST), or a contrast tube at a treatment tool distal end 13a, which is the distal end of the treatment tool 13. In addition, in the present specification, the treatment tool 13 includes a treatment instrument such as a biopsy forceps or a knife, and a wire or the like connected to the treatment instrument for operating the treatment instrument.

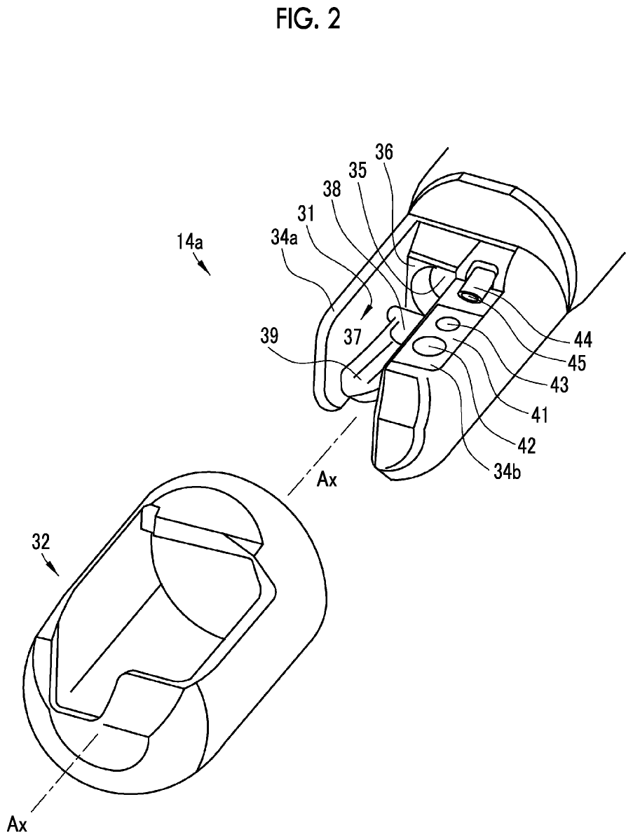

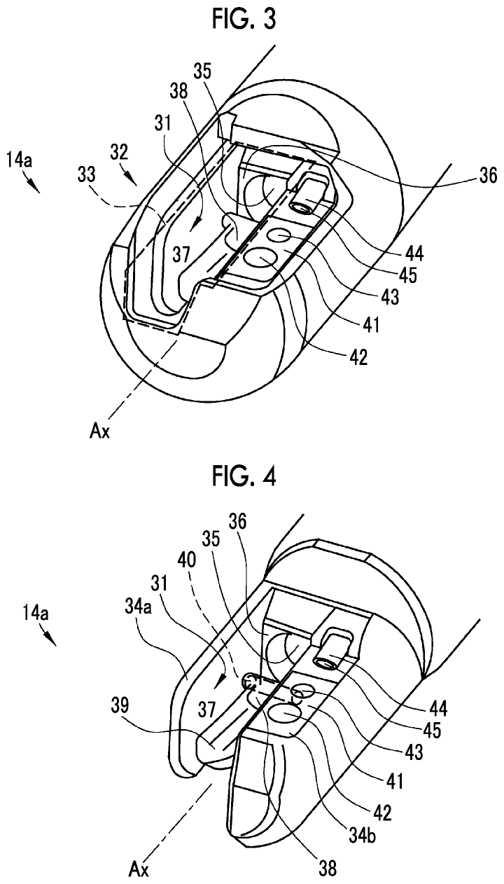

As shown in FIGS. 2 and 3, the distal end part 14a comprises a distal end part main body 31 and a distal end cap 32 that is attachably and detachably fitted onto the distal end part main body 31. The distal end cap 32 is attached to the distal end part main body 31 during a treatment using the endoscope 10. The distal end cap 32 and the distal end part main body 31 form a treatment tool protrusion port 33 provided on a side surface of the distal end part 14a and through which the treatment tool 13 protrudes. When the treatment ends, the distal end cap is removed from the distal end part main body 31 and discarded. Therefore, the distal end cap 32 is a disposable product.

The distal end part main body 31 is located at the distal end of the insertion part 14 and is made of a corrosion-resistant metal material, a plastic material, or the like. The distal end part main body 31 comprises a distal end surface 36, and the distal end surface 36 comprises a treatment tool outlet port 35. A pair of partition walls consisting of a partition wall 34a and a partition wall 34b are provided so as to face each other with the treatment tool outlet port 35 of the distal end surface 36 interposed therebetween.

The distal end part main body 31 forms an elevator housing space 37 communicating with the treatment tool outlet port 35 through the partition wall 34a, the partition wall 34b, and the distal end surface 36. The distal end cap 32 covers a part of at least the elevator housing space 37. The elevator housing space 37 is provided with a treatment tool contact portion 38 that controls an advancing direction of the treatment tool 13 by coming into contact with the treatment tool 13 led out of the treatment tool outlet port 35. In the present embodiment, the treatment tool contact portion 38 is provided on an elevator 39. The elevator 39 is provided in the elevator housing space 37. The elevator 39 elevates the treatment tool 13 of which the advancing direction is controlled by the treatment tool contact portion 38.

Therefore, the treatment tool 13 led out of the treatment tool outlet port 35 comes into contact with the treatment tool contact portion 38 of the elevator 39, and the advancing direction thereof is controlled by the treatment tool contact portion 38. By further advancing the treatment tool 13 and elevating the elevator 39, the treatment tool 13 guided by the elevator 39 protrudes outside the distal end part 14a through the treatment tool protrusion port 33.

One end of the elevator 39 is supported at, for example, an elevator support portion 40 so as to be rotationally movable with respect to the distal end part main body 31. As shown in FIG. 4, the elevator support portion 40 is a shaft in the present embodiment. Therefore, the elevator 39 is freely elevated and reclined around the elevator support portion 40. The elevator 39, which has controlled the elevated and reclined positions, controls a protruding direction of the treatment tool 13 by guiding the treatment tool 13, and fixes the treatment tool 13 by sandwiching the treatment tool 13 between the elevator 39 and the distal end part main body 31. The configurations, functions, and the like of the treatment tool contact portion 38 and of the elevator 39 will be described in detail below.

A flat portion 41 is formed on an upper part of an outer peripheral surface of the distal end part main body 31. Since a part of the outer peripheral surface is formed by the partition wall, the flat portion 41 is provided on the upper part of the partition wall 34b. The flat portion 41 is provided with an illumination unit 42 and an observation unit 43 as observation means. The illumination unit 42 and the observation unit 43 are disposed side by side in an axial direction Ax of the insertion part 14. Since the treatment tool 13 is led out of the treatment tool outlet port 35 to the outside in the axial direction Ax of the insertion part 14, the illumination unit 42 and the observation unit 43 are disposed side by side in parallel to a lead-out direction of the treatment tool 13, and the distance between the illumination unit 42 and the observation unit 43, and the treatment tool 13 is within a certain range. Therefore, the illumination and observation by the illumination unit 42 and the observation unit 43 can be performed without any problem regardless of which direction the protruding direction of the treatment tool 13 is depending on the elevated state of the treatment tool 13.

In the flat portion 41, an air and water supply nozzle 44 is provided on the operation part 15 side of the endoscope 10 with respect to the observation unit 43. In the air and water supply nozzle 44, an opening 45 through which a gas, such as air, or a liquid, such as water, is discharged is disposed toward the observation unit 43. Therefore, by jetting the gas and the liquid from the air and water supply nozzle 44, dirt and the like mainly attached to the observation unit 43 are washed.

The illumination unit 42 comprises an optical system housing chamber inside the partition wall 34b, and an illumination portion and an imaging portion are housed inside the optical system housing chamber. The inside of the partition wall 34b is not shown. The illumination portion comprises an illumination lens disposed on the optical system housing chamber side of the illumination unit 42 and a light guide disposed such that an emission end thereof faces the illumination lens. The light guide is disposed from the insertion part 14 of the endoscope 10 (see FIG. 1) to the universal cord 16 via the operation part 15, and the proximal end thereof is connected to the connector 17. With this, in a case where the connector 17 is connected to the light source device 12, irradiation light from the light source device 12 is emitted from the illumination unit 42 to a visual field region of the endoscope 10 via the light guide.

The observation unit 43 is provided with a side-view observation optical system for observing a side-view direction of the insertion part 14, and, for example, an image sensor, such as complementary metal oxide semiconductor (CMOS) or charge coupled device (CCD), is disposed at an image formation position of this side-view observation optical system. A signal cable is connected to the image sensor, and the signal cable is disposed from the insertion part 14 of the endoscope 10 (see FIG. 1) to the universal cord 16 via the operation part 15, and the proximal end thereof is connected to the connector 17. With this, in a case where the connector 17 is connected to the processor device 11, an imaging signal of a subject image obtained by the observation unit 43 is transmitted to the processor device 11 via the signal cable.

The operation part 15 (see FIG. 1) is provided with a treatment tool inlet port 18, a bending operation knob 19, and an elevating operation lever 20. The treatment tool 13 is introduced from the treatment tool inlet port 18 with the treatment tool distal end 13a at the head, inserted into the treatment tool channel installed inside the insertion part 14, and is led out of the treatment tool outlet port 35 provided in the distal end part main body 31 to the outside of the distal end part 14a.

In a case where the bending operation knob 19 is operated, an angle wire inserted and provided in the insertion part 14 is pushed and pulled, whereby the bending part 14b performs a bending motion in up, down, left, and right directions. As a result, the distal end part 14a is directed to the desired direction in the body.

The elevating operation lever 20 is rotatably provided. In a case where rotation operation of the elevating operation lever 20 is performed, a pushing and pulling operation of an elevator wire (not shown) inserted and provided in the insertion part 14 is performed in conjunction with the rotation operation of the elevating operation lever 20. By such an operation of the elevator wire, the position of the elevator 39 coupled to the distal end side of the elevator wire is changed between the reclined position and the elevated position.

In addition, an air and water supply button 46 and a suction button 47 are provided in parallel on the operation part 15. In a case where the air and water supply button 46 is operated, for example, air and water are supplied to an air and water supply tube (not shown), and air and water can be ejected from the air and water supply nozzle 44 provided in the distal end part main body 31. The air and water supply button 46 is a multi-stage push button, and, for example, air is supplied to the air and water supply tube by a one-stage operation, and water is supplied to the air and water supply tube by a two-stage operation. By operating the suction button 47, body fluid, such as blood, can be suctioned from the suction port that also serves as the treatment tool outlet port 35 via the treatment tool channel.

The processor device 11 (see FIG. 1) acquires the imaging signal output from a CCD or the like via the signal cable and performs various types of image processing to generate image data. The image data generated by the processor device 11 is displayed as an observation image on the display 21 connected to the processor device 11.

Next, the configurations, functions, and the like of the treatment tool contact portion 38 and of the elevator 39 will be described in detail. The treatment tool contact portion 38 is provided in order to control the advancing direction of the treatment tool 13 by bringing the treatment tool 13 that has come out of the treatment tool outlet port 35 into contact with the treatment tool contact portion 38. The treatment tool 13 has the axial direction Ax in the treatment tool channel as the advancing direction. However, after coming out of the treatment tool outlet port 35, the treatment tool 13 is brought into contact with the treatment tool contact portion 38, so that the advancing direction can be moderately changed while maintaining a good insertability of the treatment tool 13. The treatment tool 13 of which the advancing direction is moderately changed protrudes smoothly through the treatment tool protrusion port 33. The treatment tool contact portion 38 is provided on the path of the treatment tool 13 in the elevator housing space 37.

In addition, it is preferable that a member with which the treatment tool 13 first comes into contact after coming out of the treatment tool outlet port 35 is the treatment tool contact portion 38. First coming into contact means that, in a case where the elevator 39 is in the reclined position, the treatment tool 13 that has come out of the treatment tool outlet port 35 first comes into contact with the treatment tool contact portion 38 without coming into contact with other members. In a case where the treatment tool 13 comes into contact with a member other than the treatment tool contact portion 38, for example, the elevator 39 or the like, there may be cases where problems arise depending on situations such as the angle of contact or the like, such as the path of the treatment tool 13 being hindered and the treatment tool 13 becoming unable to advance further, or the advancing direction of the treatment tool 13 being changed to a direction other than the treatment tool protrusion port 33.

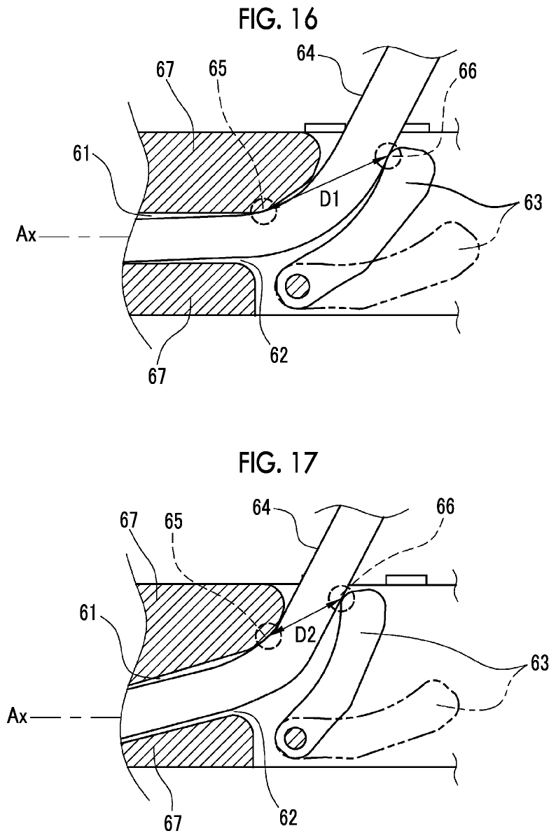

In addition, in a case where the protruding direction or the like of the treatment tool 13 is controlled by the elevator 39, by placing the elevator 39, which comes into contact with the treatment tool 13 and elevates the treatment tool 13, in the elevated position, the treatment tool 13 is bent in a direction of the treatment tool protrusion port 33. Generally, in this case, the contact point between the treatment tool 13 and the upper part of the treatment tool outlet port 35 serves as the fulcrum point (65), and the contact point between the treatment tool 13 and the elevator 39 serves as the force point (66) (see FIG. 16). By making the distance (D1) between the fulcrum point and the force point longer, it is possible to further improve the elevating force of the elevator 39, which is a force for placing the elevator 39 in the elevated position to elevate the treatment tool 13.

Therefore, it is preferable to install the treatment tool contact portion 38 based on a position and a shape where the treatment tool contact portion 38 serves as the fulcrum point and based on a position and a shape where the elevator 39 serves as the force point at a position where the distance from the fulcrum point is longer, in a case of bending the treatment tool 13 in the direction of the treatment tool protrusion port 33 by placing the elevator 39 in the elevated position. As a result, a small amount of force is sufficient to operate the elevator 39 to elevate the treatment tool 13. In addition, this facilitates control of the lead-out direction of the treatment tool 13. Accordingly, the elevating force of the elevator 39 can be improved.

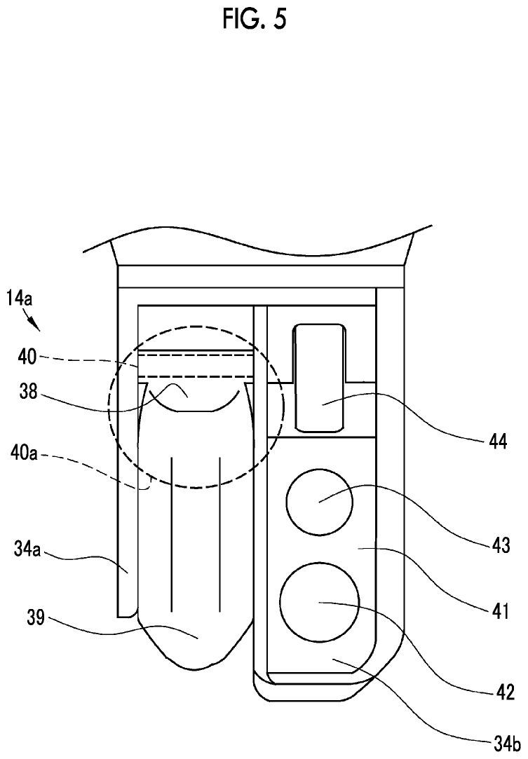

As shown in FIG. 5, it is preferable that the treatment tool contact portion 38 is provided in a vicinity 40a of the elevator support portion 40. The vicinity 40a of the elevator support portion 40 is, in a case where the treatment tool contact portion 38 is provided at this position, a position where the treatment tool 13 that has come out of the treatment tool outlet port 35 first comes into contact with the treatment tool contact portion 38. In addition, the vicinity 40a of the elevator support portion 40 may be, in a case where the elevator housing space 37 is divided into two in the axial direction Ax, a part on the elevator support portion 40 side. By providing the treatment tool contact portion 38 in the vicinity 40a of the elevator support portion 40, the treatment tool 13 that has come out of the treatment tool outlet port 35 first comes into contact with the treatment tool contact portion 38, and there is no risk that the treatment tool 13 may come into contact with other members and the like and that the leading out of the treatment tool 13 may be hindered, so that the treatment tool contact portion 38 can effectively function and a good insertability of the treatment tool 13 can be obtained.

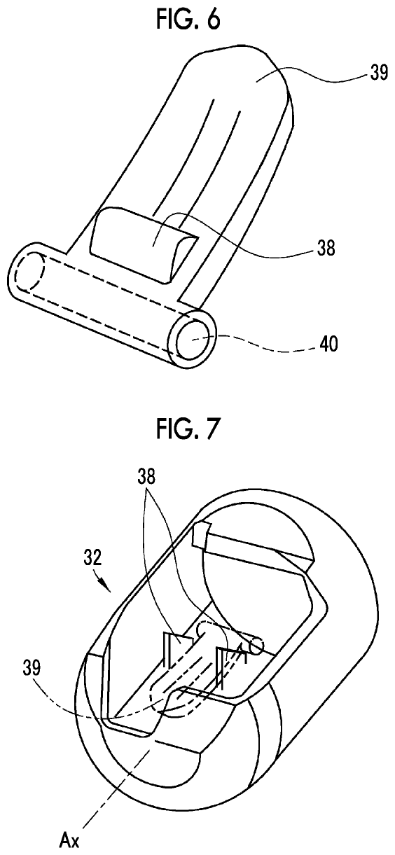

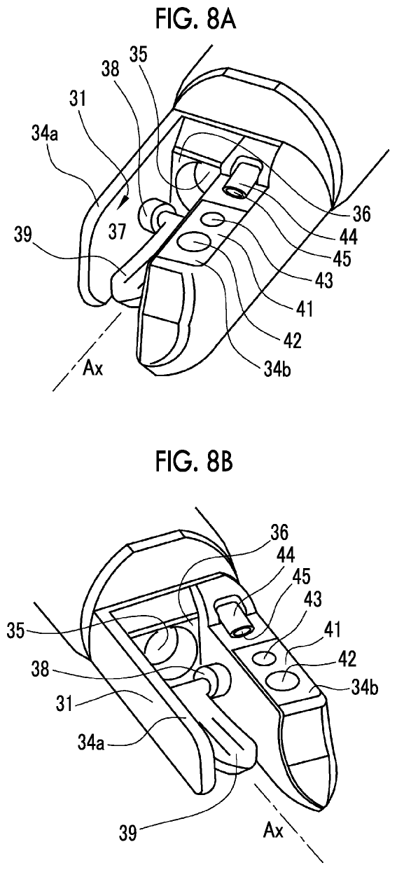

Specific examples of a case where the treatment tool contact portion 38 is provided include providing the treatment tool contact portion 38 on the elevator 39 as shown in FIG. 6, providing the treatment tool contact portion 38 on the distal end cap 32 as shown in FIG. 7, or providing the treatment tool contact portion 38 on the distal end part main body 31 as shown in FIGS. 8A and 8B. In any case, in a case where the treatment tool 13 that has come out of the treatment tool outlet port 35 advances through the elevator housing space 37, the treatment tool 13 comes into contact with the treatment tool contact portion 38 to appropriately change the advancing direction of the treatment tool 13 to the treatment tool protrusion port 33. Which one to select can be determined depending on the type, shape, purpose of treatment, or the like of the treatment tool 13.

In the present embodiment, the treatment tool contact portion 38 is provided on the elevator 39 (see FIG. 6). In a case where the treatment tool contact portion 38 is provided on the elevator 39, all treatment tools 13 can be preferably used as a target regardless of the type of the treatment tool 13.

In addition, in a case where the treatment tool contact portion 38 is provided on the elevator 39, it is preferable that the treatment tool contact portion 38 is provided in a region within half of the elevator 39 on the elevator support portion 40 side in the longitudinal direction. It is more preferable that the treatment tool contact portion 38 is provided in a region within ¼ of the elevator 39 on the elevator support portion 40 side in the longitudinal direction. By providing the treatment tool contact portion 38 in the above-described region, the treatment tool 13 that has come out of the treatment tool outlet port 35 can come into contact with the treatment tool contact portion 38 without coming into contact with other members and the like, so that it is possible to appropriately control the advancing direction of the treatment tool 13.

The treatment tool contact portion 38 may be provided on the distal end cap 32 (see FIG. 7). In a case where the treatment tool contact portion 38 is provided on the distal end cap 32, it is preferable that the treatment tool 13 that has come out of the treatment tool outlet port 35 is provided at a position where the treatment tool 13 can come into contact with the treatment tool contact portion 38 without coming into contact with other members and the like. In addition, the treatment tool 13 comes into contact with the treatment tool contact portion 38 to change the advancing direction and then comes into contact with the elevator 39 in response to the movement of the elevator 39, and the position or the like of the treatment tool 13 is controlled. Therefore, it is preferable that the treatment tool contact portion 38 is provided at a position where the movement of the elevator 39 is not hindered. In FIG. 7, the distal end cap 32 comprises two treatment tool contact portions 38, but a shape may be employed in which one or three or more are provided.

In addition, since the distal end cap 32 is mounted on the distal end part main body 31, a plurality of the distal end caps 32 in which the installation position of the treatment tool contact portion 38 is changed or the shape, number, position, or the like of the treatment tool contact portion 38 is changed are prepared and then, for example, an appropriate distal end cap 32 can be selected according to the shape, type, or the like of the treatment tool 13 and mounted on the distal end part main body 31. Therefore, an appropriate treatment tool contact portion 38 can be provided for a plurality of types of treatment tools 13 by an easy method such as selecting the distal end cap 32 and mounting the distal end cap 32 on the distal end part main body 31.

The treatment tool contact portion 38 may be provided on the distal end part main body 31 (see FIGS. 8A and 8B). In a case where the treatment tool contact portion 38 is provided on the distal end part main body 31, similarly to a case where the treatment tool contact portion 38 is provided on the distal end cap 32, it is preferable that the treatment tool contact portion 38 is provided at a position where the treatment tool 13 that has come out of the treatment tool outlet port 35 can come into contact with the treatment tool contact portion 38 without coming into contact with other members and the like, and that the treatment tool contact portion 38 is provided at a position where the movement of the treatment tool 13 is not hindered. As shown in FIGS. 8A and 8B, the distal end part main body 31 comprises two treatment tool contact portions 38. Specifically, on the distal end part main body 31, one treatment tool contact portion 38 is provided in a state of being in contact with the partition wall 34a, and one treatment tool contact portion 38 is provided in a state of being in contact with the partition wall 34b. As shown in FIGS. 8A and 8B, these treatment tool contact portions 38 may also be provided with a function as the elevator support portion 40. On the distal end part main body 31, one or three or more treatment tool contact portions 38 may be provided.

In a case where the treatment tool contact portion 38 is provided on the distal end cap 32 or the distal end part main body 31, the treatment tool contact portion 38 does not move in conjunction with the rotational motion in which the elevator 39 is moved between the reclined position and the elevated position. Therefore, there is no risk that the treatment tool contact portion 38 may interfere during the rotational motion such as when the elevator 39 is elevated, which is preferable.

It is preferable that the treatment tool contact portion 38 is a protrusion. The protrusion refers to a part protruding from the periphery, and the periphery is the periphery of a location where the treatment tool contact portion 38 is provided. Specifically, the periphery refers to an installation surface of the treatment tool contact portion 38, such as the elevator 39, the distal end cap 32, or the distal end part main body 31.

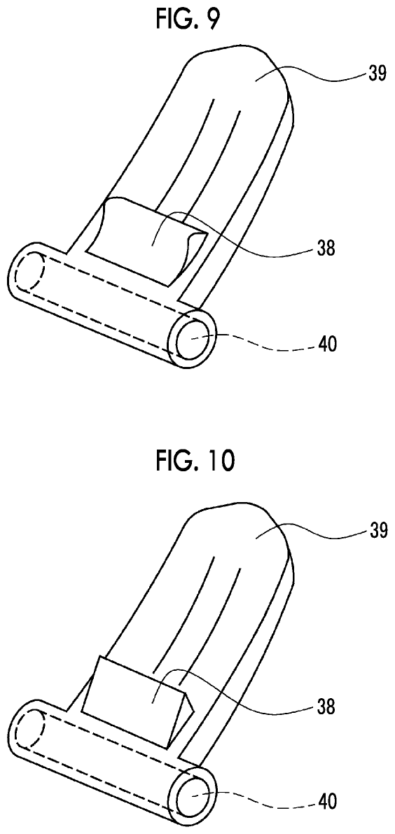

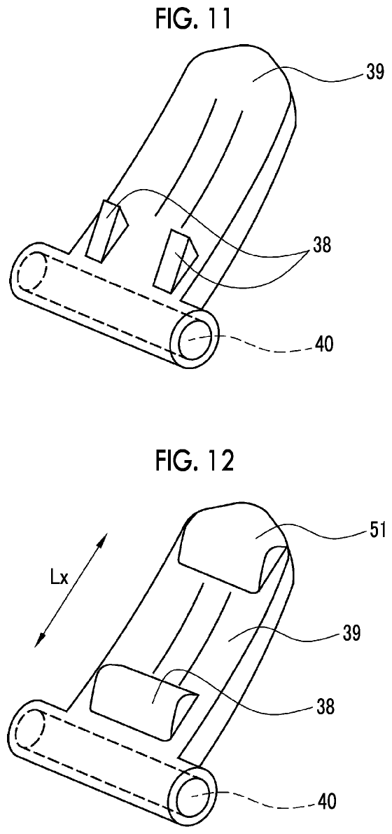

It is preferable that the shape of the treatment tool contact portion 38 is a shape in which, in a case where the treatment tool 13 that has come out of the treatment tool outlet port 35 first comes into contact with the treatment tool contact portion 38, the advancing direction of the treatment tool 13 can be moderately controlled without hindering the advancement of the treatment tool 13. For example, it is preferable that the shape of the treatment tool contact portion 38 is a shape in which the height, angle, or the like of the protrusion is adjusted such that a part close to the treatment tool outlet port 35 is gentle and the treatment tool 13 gradually moves toward a desired advancing direction. As shown in FIG. 9, in the present embodiment, the treatment tool contact portion 38 can be provided as a protrusion having a gentle mountain-like shape at a position close to the elevator support portion 40 of the elevator 39, and can be provided as a protrusion having a shape with a steep angle at a position far from the elevator support portion 40. In the treatment tool contact portion 38 which is a protrusion having a mountain-like shape, the angle of the mountain is set to an angle at which the treatment tool 13 can smoothly and easily advance when the treatment tool 13 is advanced, according to the type of the treatment tool 13, or the position, shape, or the like of the treatment tool channel. Further, as shown in FIG. 10, the treatment tool contact portion 38 may have a triangular prism shape.

It is preferable that the distance between the fulcrum point and the force point of the treatment tool contact portion 38 is made longer in a shape, an installation position, or the like in a case where the elevator 39 is placed in the elevated position. Therefore, it is preferable that the shape of the treatment tool contact portion 38 is made such that the angle of the part close to the treatment tool outlet port 35 is made gentle, the advancement of the treatment tool 13 is guided at this part, and a position where the treatment tool contact portion 38 is highest as compared with the periphery, that is, the position serving as the force point, is made farther from the treatment tool outlet port 35. Therefore, in a case where the elevator support portion 40 of the elevator 39 is a shaft, it is preferable that the treatment tool contact portion 38 is installed in a triangular prism shape when viewed from a direction of the shaft such that the contact surface of the treatment tool 13 is a widest side surface of the triangular prism (see FIG. 10). This is because the highest ridgeline part of the triangular prism far from the elevator support portion 40 can be used as the force point.

A plurality of the treatment tool contact portions 38 may be provided. The number of the treatment tool contact portions 38 can be determined depending on the type, shape, size or thickness, function, or the like of the treatment tool 13. As shown in FIG. 11, for example, in order to lead out a relatively thick treatment tool 13, two triangular prism-shaped protrusions can be installed at both ends of the elevator 39 in a width direction at positions in the vicinity of the elevator support portion 40 of the elevator 39.

In a case where the plurality of treatment tool contact portions 38 are provided, a thick treatment tool 13 can be targeted. Since the thick treatment tool 13 tends to have a strong rigidity, the treatment tool 13 may be caught on the central region or the like in the longitudinal direction of the elevator 39 as the treatment tool 13 advances, and the advancement of the treatment tool 13 is likely to be hindered, in a case where the treatment tool contact portion 38 is not provided. Therefore, by providing the plurality of treatment tool contact portions 38, even a treatment tool 13 having a problem in insertability, such as a thick treatment tool 13, can obtain a good insertability without affecting others.

In addition, in a case where the plurality of treatment tool contact portions 38 are provided, it is possible to set in detail positions, shapes, or the like in a case where the plurality of treatment tool contact portions 38 are provided in conformity with the shape or the like of the treatment tool 13. For example, depending on the shape or the like of the treatment tool 13, by providing the plurality of treatment tool contact portions 38 in an asymmetric manner on the elevator 39, by providing the plurality of treatment tool contact portions 38 having shapes different from each other, or the like, the optimum treatment tool contact portion 38 having a high degree of freedom in installation and conforming to the treatment tool 13 can be installed.

In the present embodiment, the elevator 39 is provided with the treatment tool contact portion 38, but the shape, type, installation position, or the like of the treatment tool contact portion 38 can also be applied to a case (see FIG. 7, 8A, or 8B) where the treatment tool contact portion 38 is provided on the distal end cap 32 or the distal end part main body 31. Therefore, even in a case where the treatment tool contact portion 38 is provided on the distal end cap 32 or the distal end part main body 31, the treatment tool contact portions 38 having various shapes can be provided, and the plurality of treatment tool contact portions 38 can be provided at locations different from each other. In addition, in a case where the plurality of treatment tool contact portions 38 are provided, the treatment tool contact portions 38 may be provided on another member. For example, in a case where the plurality of treatment tool contact portions 38 are provided, some of the treatment tool contact portions 38 may be provided on the distal end cap 32 and some thereof may be provided on the distal end part main body 31.

The elevator 39 may be provided with a distal end part protrusion in a half region opposite to the elevator support portion 40 side in the longitudinal direction of the elevator 39. The half region opposite to the elevator support portion 40 side refers to, in a case where a half position of the elevator 39 in a longitudinal direction Lx is determined, a region opposite to the elevator support portion 40 side from this half position. This region is a region of the distal end of the elevator 39, and is, in a case where the elevator 39 is placed in the elevated position, a region that comes into contact with the wire or the like of the treatment tool 13 protruding through the treatment tool protrusion port 33, that elevates the treatment tool 13 using the contacted portion as the above-described force point, that fixes the treatment tool 13 by sandwiching the treatment tool 13 with the distal end part main body 31. A protrusion provided in this region is referred to as the distal end part protrusion, and the distal end part protrusion is provided, so that the treatment tool 13 protruding outside the distal end part can be more firmly fixed by the distal end part main body 31 and the distal end part protrusion. In addition, since the treatment tool 13 is thin, the treatment tool 13 can be more reliably fixed even in a case where it is difficult to fix the treatment tool 13.

As shown in FIG. 12, a distal end part protrusion 51 is a protrusion provided in the half region opposite to the elevator support portion 40 side in the longitudinal direction Lx of the elevator 39. It is preferable that the shape or the installation position of the distal end part protrusion 51 is a shape or an installation position where the advancement of the treatment tool 13 is not hindered in a case where the treatment tool 13 is led out. In a case where the distal end part protrusion 51 is provided when the treatment tool contact portion 38 is not provided, there is a risk that the distal end part protrusion 51 may hinder the advancement of the treatment tool 13 in a case where the treatment tool 13 is led out. However, in a case where the distal end part protrusion 51 is provided after the treatment tool contact portion 38 is provided, the distal end part protrusion 51 can be installed with a relatively high degree of freedom in the shape, installation position, or the like, and there is little risk of hindering the advancement of the treatment tool 13 in a case where the treatment tool 13 is led out. This is because the advancing direction of the treatment tool 13 is controlled in advance by the treatment tool contact portion 38. Therefore, it is preferable that both the treatment tool contact portion 38 and the distal end part protrusion 51 are provided on the elevator 39.

In addition, it is preferable that the elevator 39 comprises the distal end part protrusion 51 at a position where the treatment tool 13 comes into contact in a case where the elevator 39 is elevated to the elevated position, and that the treatment tool contact portion 38 controls the advancing direction of the treatment tool 13 to come into contact with the distal end part protrusion 51 in a case in which the elevator 39 is in the reclined position. In the elevator 39 provided with the distal end part protrusion 51, a case where the treatment tool distal end 13a that has come out of the treatment tool outlet port 35 comes into contact with the distal end part protrusion 51 and a case where the treatment tool distal end 13a does not come into contact with the distal end part protrusion 51 are considered. However, as will be described below, in any case, in a case where the elevator 39 moves to the elevated position, the distal end part protrusion 51 of the elevator 39 comes into contact with the treatment tool 13, and this contact point serves as the force point, so that the treatment tool 13 can be easily elevated by the distal end part protrusion 51.

It is preferable that, in a case where the treatment tool 13 comes into contact with the treatment tool contact portion 38 and then is further advanced and the elevator 39 lifts the treatment tool 13, the elevator 39 and the treatment tool 13 come into contact with each other only at the distal end part protrusion 51. In particular, it is preferable for the elevator 39 and the treatment tool 13 to come into contact with only the distal end part protrusion 51 in a case where the elevator 39 is at an angle at which a force is required to lift the treatment tool 13, that is, at a position close to the reclined state. This is because the distal end part protrusion 51 serves as the force point away from the fulcrum point described above and the treatment tool 13 can be elevated with a smaller amount of force.

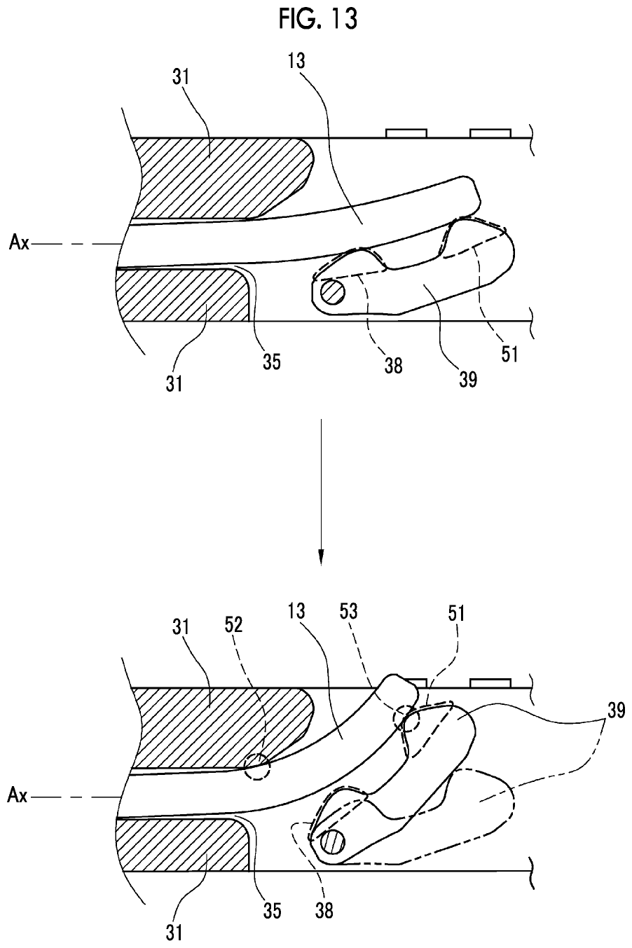

As shown in FIG. 13, specifically, in a case where the distal end of the treatment tool 13 led out of the treatment tool outlet port 35 comes into contact with the treatment tool contact portion 38, is further led out to the treatment tool protrusion port 33, and comes into contact with the distal end part protrusion 51, by lifting the elevator 39 in this state by, for example, about one third of the maximum elevated position, the treatment tool 13 is separated from the treatment tool contact portion 38. In addition, with the elevating of the elevator 39, a location where the treatment tool 13 comes into contact with the distal end part main body 31 serves as a fulcrum point 52, and a location where the treatment tool 13 comes into contact with the distal end part protrusion 51 serves as a force point 53. That is, the elevator 39 and the treatment tool 13 come into contact with each other only at the force point 53. Therefore, the elevator 39 comes into contact with the treatment tool 13 at a location away from the fulcrum point 52 to lift the treatment tool 13, so that the treatment tool 13 can be elevated with a smaller amount of force.

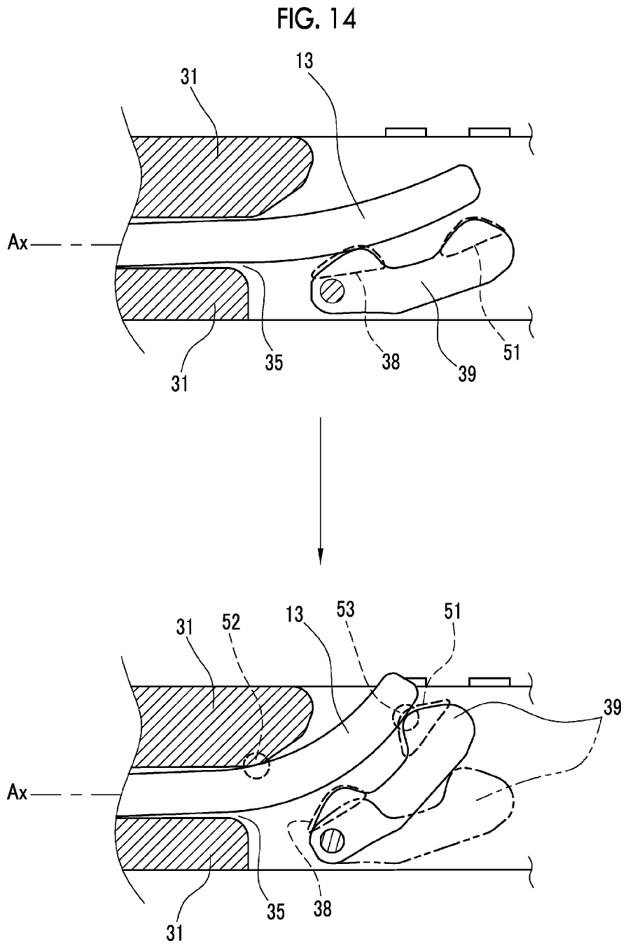

As shown in FIG. 14, in a case where the distal end of the treatment tool 13 led out of the treatment tool outlet port 35 comes into contact with the treatment tool contact portion 38, is further led out to the treatment tool protrusion port 33, and does not come into contact with the distal end part protrusion 51, by lifting the elevator 39 in this state by, for example, about one third of the maximum elevated position, the distal end part protrusion 51 comes into contact with the treatment tool 13, and the treatment tool 13 is separated from the treatment tool contact portion 38. As in the case of FIG. 13, with the elevating of the elevator 39, the elevator 39 and the treatment tool 13 comes into contact with each other only at the force point 53, the elevator 39 comes into contact with the treatment tool 13 at a location away from the fulcrum point 52, and the treatment tool 13 is elevated, so that the treatment tool 13 can be elevated with a smaller amount of force.

In this way, in order to ensure that the distal end part protrusion 51 is provided at a position where the treatment tool 13 comes into contact in a case where the elevator 39 is elevated to the elevated position, the shape, position, or the like of the treatment tool contact portion 38 or of the distal end part protrusion 51 can be adjusted. Further, in this case, the treatment tool contact portion 38 may be provided on the elevator 39, or may be provided on the distal end cap 32 or on the distal end part main body 31 as in the present embodiment.

In addition, it is preferable that the endoscope 10 is a disposable product in part or in whole. In particular, it is preferable that a part where there is a risk of infection in a case where a reusable product is used is made a disposable product. Therefore, it is preferable that the distal end part 14a is made a disposable product, and in particular, the distal end part main body 31, the elevator 39, the distal end cap 32, the treatment tool contact portion 38, and the like are preferably a disposable product. With this, even in the elevator 39, the distal end part main body 31, or the distal end cap 32, which is provided with the treatment tool contact portion 38, or the elevator 39 provided with the distal end part protrusion 51, it is not necessary to consider cleaning of the uneven part, so that the treatment tool contact portion 38, the distal end part protrusion 51, or the like can be designed and disposed with a high degree of freedom. Therefore, the endoscope 10 can be effectively used in a case where it is a disposable product.

The entire insertion part 14 other than the distal end part 14a may be a disposable product, or the entire endoscope 10 may be a disposable product. In these cases, components with no risk of infection in a case of reuse, for example, a part or all of the illumination unit 42 provided in the partition wall 34b, a part or all of the observation unit 43, or the like may be reused.

In a case where a part or all of the endoscope 10 is a disposable product, the endoscope 10 is preferably a side-viewing endoscope. The side-viewing endoscope is used as a duodenal endoscope and is used for endoscopic retrograde cholangiopancreatography (ERCP) of inserting the endoscope 10 into the subject, directly inserting a thin catheter into a bile duct or a pancreatic duct in the duodenum, injecting a contrast agent, and capturing an X-ray image. In this case, in a case where the endoscope is a reusable product, the transmission of the multidrug-resistant bacteria becomes a problem. Therefore, in a case where the endoscope 10 is a disposable product, both the insertability and the elevating force can be improved, the treatment tool 13 to be used for the ERCP can be appropriately led out, and there is no risk of infection or the like, so that the endoscope 10 can be suitably used as a duodenal endoscope.

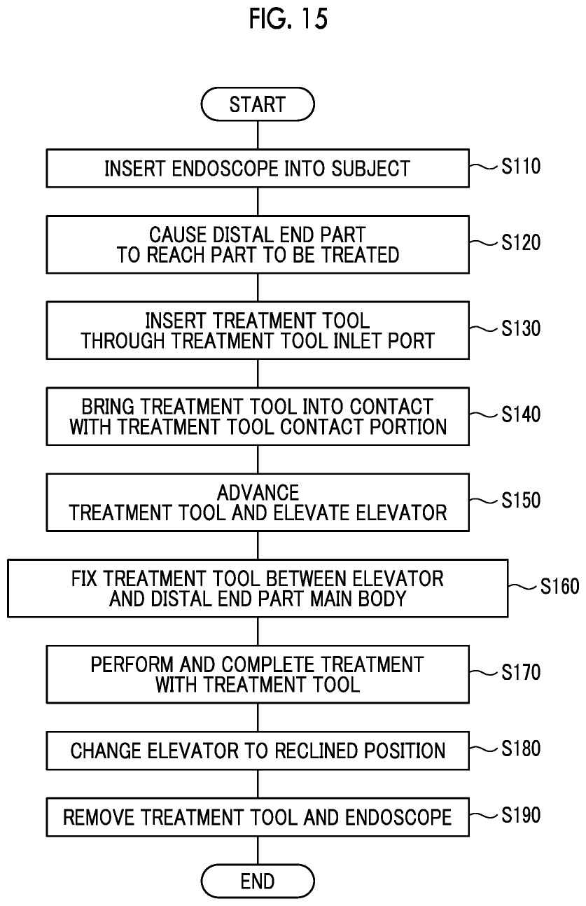

Next, the operation of the endoscope 10 will be described with reference to the flowchart of FIG. 15. In a case where the treatment is performed using the endoscope 10, the endoscope 10 is inserted into the subject (step ST110), and the distal end part 14a is advanced to the part to be treated while checking the image of the endoscope 10 appearing on the display 21 (step ST120). The treatment tool 13 corresponding to the treatment is inserted through the treatment tool inlet port 18 (step ST130). The treatment tool 13 comes out of the treatment tool outlet port 35 and comes into contact with the treatment tool contact portion 38 (step ST140), and control to change the advancing direction of the treatment tool 13 from the axial direction Ax to the direction of the treatment tool protrusion port 33 is performed. The treatment tool 13 comes into contact with the treatment tool contact portion 38, so that the advancing direction of the treatment tool 13 can be easily changed without hindering the advancement of the treatment tool 13. In a case of leading out the treatment tool 13, the elevator 39 is placed in the reclined position in advance.

After the treatment tool 13 comes into contact with the treatment tool contact portion 38, the treatment tool 13 is further advanced and the elevator wire is operated to elevate the elevator 39 (step ST150). As a result, the elevator 39 changes from the reclined position to the elevated position, and the treatment tool 13 supported by the elevator 39 is also elevated in response to the positional change of the elevator 39. In a case where the treatment tool 13 is elevated, the treatment tool contact portion 38 facilitates the operation of the elevator 39 using the elevator wire because the lever principle is used after the fulcrum point and the force point are separated from each other. The protruding direction, the protruding length, and the like of the treatment tool 13 are adjusted, and the elevator 39 is elevated to sandwich and fix the wire part and the like of the treatment tool 13 between the elevator 39 and the distal end part main body 31 (step ST160). Treatment is performed with the treatment tool 13 whose position is fixed and stable (step ST170). In a case where the treatment is completed, the elevator 39 is changed to the reclined position (step ST180), the treatment tool 13 is returned, and the endoscope 10 is removed (step ST190). With the above, the treatment with the treatment tool 13 using the endoscope 10 ends.

Since the endoscope 10 is configured as described above and the treatment tool contact portion 38 is provided at the distal end part 14a of the endoscope 10, the treatment tool 13 can be easily elevated without hindering the advancement of the treatment tool 13 in a case where the treatment tool 13 protrudes through the elevator 39 at the distal end part 14a of the endoscope 10. Therefore, the endoscope 10 is excellent in both the insertability and the lifting force of the treatment tool 13. The endoscope 10 is excellent in both the insertability of the treatment tool and the lifting force and is suitable as a duodenal endoscope which is a side-viewing endoscope. In particular, by making the endoscope 10 or the distal end part 14a a disposable product, a duodenal endoscope with no risk of infection is obtained, which is particularly suitable.

The endoscope 10 provided with the treatment tool contact portion 38 of the embodiment of the present invention is an endoscope 10 used for observing or treating the inside of the subject, but can also be suitably used for various devices, such as other endoscopes using the treatment tool 13.

### EXPLANATION OF REFERENCES

2: endoscope system
10: endoscope
11: processor device
12: light source device
13, 64: treatment tool
13a: treatment tool distal end
14: insertion part
14a: distal end part
14b: bending part
14c: flexible tube part
15: operation part
16: universal cord
17: connector
18: treatment tool inlet port
19: bending operation knob
20: operation lever
21: display
31, 67: distal end part main body
32: distal end cap
33: treatment tool protrusion port
34a, 34b: partition wall
35, 62: treatment tool outlet port
36: distal end surface
37: elevator housing space
38: treatment tool contact portion
39, 63: elevator
40: elevator support portion
40a: vicinity
41: flat portion
42: illumination unit
43: observation unit
44: air and water supply nozzle
45: opening
46: air and water supply button
47: suction button
51: distal end part protrusion
52, 65: fulcrum point
53, 66: force point
61: treatment tool insertion channel
Ax: axial direction
Lx: longitudinal direction
D1, D2: distance
ST110 to ST190: step

What is claimed is:

1. An endoscope comprising:
an insertion part to be inserted into a subject;
a distal end part main body that is located at a distal end of the insertion part, has a distal end surface provided with a treatment tool outlet port, and forms an elevator housing space communicating with the treatment tool outlet port;
a treatment tool contact portion, that is a member provided in the elevator housing space and comes into contact with a distal end of a treatment tool that has come out of the treatment tool outlet port, to control an advancing direction of the treatment tool; and
an elevator that is provided in the elevator housing space and elevates the treatment tool of which the advancing direction is controlled by the treatment tool contact portion.

2. The endoscope according to claim 1,
wherein one end of the elevator is supported at an elevator support portion so as to be rotationally movable with respect to the distal end part main body, and
the treatment tool contact portion is provided in a vicinity of the elevator support portion.

3. The endoscope according to claim 2,
wherein the treatment tool contact portion is provided in a half region of the elevator on a side of the elevator support portion in a longitudinal direction.

4. The endoscope according to claim 1, further comprising:
a cap that is attached to the distal end part main body and covers at least a part of the elevator housing space,
wherein the treatment tool contact portion is provided on the cap.

5. The endoscope according to claim 1,
wherein the treatment tool contact portion is provided on the distal end part main body.

6. The endoscope according to claim 1,
wherein the treatment tool contact portion is a protrusion.

7. The endoscope according to claim 1,
wherein the treatment tool contact portion has a triangular prism shape.

8. The endoscope according to claim 1,
wherein a plurality of the treatment tool contact portions are provided.

9. The endoscope according to claim 2,
wherein the elevator includes a distal end part protrusion in a half region opposite to a side of the elevator support portion in a longitudinal direction.

10. The endoscope according to claim 9,
wherein the treatment tool contact portion controls the advancing direction of the treatment tool to a direction of the distal end part protrusion in a case where the elevator is in a reclined position, and
the elevator includes the distal end part protrusion at a position where the treatment tool comes into contact in a case where the elevator is elevated to an elevated position.

11. The endoscope according to claim 1,
wherein a part or all of the endoscope is disposable.
